(19) United States
(12) Patent Application Publication
(10) Pub. No.: US 2026/0345868 A1
(43) Pub. Date: Nov. 5, 2026

(54) BIOPSY MARKER UTILIZING LIGHT CURED BIOABSORBABLE GELS TO SUPPORT TISSUE

(71) Applicant: BARD PERIPHERAL VASCULAR, INC., Franklin Lakes, NJ (US)

(72) Inventors: Zachary Willis, Mesa, AZ (US); Katherine Huffer, Chandler, AZ (US)

(73) Assignee: BARD PERIPHERAL VASCULAR, INC., Franklin Lakes, NJ (US)

(21) Appl. No.: 19/488,170

(22) PCT Filed: May 31, 2023
(86) PCT No.: PCT/US2023/024017

§ 371 Date: Nov. 26, 2025

(51) Int. Cl. A61L 27/54; A61K 31/42; A61K 31/43; A61K 31/431; A61K 31/4709; A61K 31/496; A61K 31/5383; A61K 31/545; A61K 31/546; A61K 31/65; A61K 31/7048; A61K 31/7052; A61K 45/06; A61L 27/18; A61L 27/22; A61L 27/52

(52) U.S. Cl. CPC A61L 27/54; A61K 31/42; A61K 31/43; A61K 31/431; A61K 31/4709; A61K 31/496; A61K 31/5383; A61K 31/545; A61K 31/546; A61K 31/65; A61K 31/7048; A61K 31/7052; A61K 45/06; A61L 27/18; A61L 27/227; A61L 27/52; A61L 2300/406; A61L 2400/06

(57) ABSTRACT

The present disclosure concerns bioabsorbable gels that are formed in situ within a cavity or space in a subject by light-initiated cross-linking as a precursor solution is applied into the desired space. In aspects, the precursor solution includes a tracking material and/or therapeutic or other component therein to provide a benefit to the subject as the gel degrades. Also contemplated are devices for application of the precursor solution that initiate cross-linking as the solutions fills the desired space.

### TECHNICAL FIELD

The present specification generally relates to photo-reactive biobabsorbable gels that once cured by light provide a marker as tissue support during healing.

### BACKGROUND

Biopsies and lumpectomies are important procedural events to assess for and remove diseased tissue, such as cancerous or pre-cancerous tissue. While detecting and removing diseased tissue is of great importance to the patient, the procedures provides some challenging after-effects. The removal of tissue itself poses the challenge of creating a void within the patient. While the body can eventually rebuild most tissue, the process can be lengthy and skin over the site can dimple. Further, the area of tissue removal requires prolonged monitoring to assess that the surrounding tissue remains healthy. In healed tissue however, it can be challenging to identify the exact area where the procedure occurred. Thus an important aspect of these procedures is to mark the exact location of tissue removal, visible under X-ray, ultraviolet light, magnetic resonance imaging, or ultrasound.

### SUMMARY

A 1^{st} aspect of the present disclosure, either alone or in combination with any other aspect herein, concerns a photo-reactive precursor solution of a bioabsorbable gel comprising a monomer and a photo-initiator.

A 2^{nd} aspect of the present disclosure, either alone or in combination with any other aspect herein, concerns the photo-reactive precursor solution of the 1^{st} aspect, further comprising an oligomer selected from polyethyleneimine (PEI), poly-lysine, and serum albumin.

A 3^{rd} aspect of the present disclosure, either alone or in combination with any other aspect herein, concerns the photo-reactive precursor solution of the 1^{st} aspect, wherein the monomer is selected from methacrylated gelatin, polyethylene glycol diacrylate (PEGDA), or a combination thereof.

A 4^{th} aspect of the present disclosure, either alone or in combination with any other aspect herein, concerns the photo-reactive precursor solution of the 3^{rd} aspect, further comprising polyethylene glycol (PEG), polylactic acid (PLA), polyglycolic acid (PGA), polylactic-co-glycolic acid (PLGA), polycaprolactone (PCL), aloe vera gel, hyaluronic acid (HA), sodium hyaluronate, or a combinations of two or more thereof.

A 5^{th} aspect of the present disclosure, either alone or in combination with any other aspect herein, concerns the photo-reactive precursor solution of any of the 1^{st} to 4^{th} aspects, further comprising an a-hydroxy carboxylic acid, a dioxepanone, a dioxanones, a lactide, a lactone, a carbonate, a glycolide, p-dioxanone, γ-caprolactone, or a combination of two or more thereof.

A 6^{th} aspect of the present disclosure, either alone or in combination with any other aspect herein, concerns the photo-reactive precursor solution of any of the 1^{st} to 5^{th} aspects, further comprising a tracker material.

An 7^{th} aspect of the present disclosure, either alone or in combination with any other aspect herein, concerns the photo-reactive precursor solution of the 6^{th} aspect, wherein the tracker material is selected from titanium, nitinol, polyvinyl acetate (PVA), PEG, stainless steel, or combinations thereof.

A 8^{th} aspect of the present disclosure, either alone or in combination with any other aspect herein, concerns the photo-reactive precursor solution of any of the 1^{st} to 7^{th} aspects, wherein the monomer comprises 0.1 to 70 percent by weight of the precursor solution.

A 9^{th} aspect of the present disclosure, either alone or in combination with any other aspect herein, concerns the photo-reactive precursor solution of any of the 1^{st} to 8^{th} aspects, wherein the photo-initiator is chosen from riboflavin, dimethylaminoethyl methacrylate (DMAEMA), lithium acylphosphinate (LAP), 2,2-dimethoxy-1,2-diphenylethan-1-one, 2-hydroxy-2-methyl-1-phenylpropanone, 1-hydroxy-cyclohexylphenylketone, benzophenone, isopropyl thioxanthone, 2-ethylhexyl-(4-N, N-dimethyl amino) benzoate, ethyl-4-(dimethylamino) benzoate, azobisisobutyronitrile, benzoyl peroxide, camphorquinone, 2-hydroxy-2-methyl propiophenone, 2-hydroxy-4′-(2-hydroxyethoxy)-2-methyl propiophenone, methobenzoyl formate, diphenyl (2,4,6-trimethylbenzoyl)-phosphine oxide, phosphine oxide phenyl bis (2,4,69 trimethyl benzoyl), 2-methyl-1-[4-(methylthio) phenyl]-2-(4-morpholiny1)-1-propanone, 2-Benzyl-2-(dimethylamino)-1-[4-(4-morpholiny1) phenyl]-1-butanone, 2-Dimethylamino-2-(4-methyl-benzyl)-1-(4-morpholin-4-yl-phenyl)-butan-1-one, Bis(.eta.5-2,4-cylcopentadien-1-yl)-bis(2,6-difluoro-3-(1H-pyrrol-1-yl)-Phenyl)titanium, 2-Ethyl Anthraquinone, 2,4-diethyl thioxanthone, Benzil Dimethyl Ketal, 4-chloro Benzophenone, methyl-2-benzoylbenzoate, 4-phenyl Benzophenone, 2,2′-Bis(2-Chlorophenyl)-4,4′, 5,5′-Tetraphenyl-1,2′-Bi-imidazole, 2,2′,4-tris(2-chlorophenyl)-5-(3,4-dimethoxyp-enly)-4′5′-diphenyl-1,1′-biimidazole, 4-phenoxy-2.′2′-Dichloro Acetophenone, Ethyl-4-(dimethylamino)Benzoate, isoamyl 4-(Dimethylamino) benzoate, 2-Ethyl Hexyl-4-(dimethylamino) Benzoate, 4,4′Bis(diethylamino) Benzophenone, 4-(4′-methylphenylthio)-benzophenone, 1,7-Bis(9-acridinyl)heptane, N-phenyl glycine, a ruthenium metal complex and sodium persulfate, Irgacure 2959, Irgacure 184, Irgacure 651, Irgacure 369, Irgacure 907, monoacylphosphine (TPO), TPO-sodium or lithium, bisacylphosphine oxide or slats thereof, VA-086, eosin-Y, riboflavin, WSPI, BDEA, P2CK, rose Bengal, erythrosine, diphenyliodonium hexafluorophosphate, bis-(4-t-butylphenyl) iodonium hexafluorophosphate, N-methyldiethanolamine, or a combinations thereof.

A 10^{th} aspect of the present disclosure, either alone or in combination with any other aspect herein, concerns the photo-reactive precursor solution of any of the 1^{st} to 9^{th} aspects, wherein the photo-initiator comprises between 0.01 and 0.4% w/v of the precursor solution.

An 11^{th} aspect of the present disclosure, either alone or in combination with any other aspect herein, concerns the photo-reactive precursor solution of any of the 1^{st} to 10^{th} aspects, further comprising an accelerator.

A 12^{th} aspect of the present disclosure, either alone or in combination with any other aspect herein, concerns the photo-reactive precursor solution of the 11^{th} aspect, wherein the accelerator is selected from diphenyliodonium, iodonium salt(s), thioxantone, iodine salt, dialkyl aminobenzoic acid, benzyl alcohol(s), tetramethylethylenediamine (TEMED), or a combination thereof.

A 13^{th} aspect of the present disclosure, either alone or in combination with any other aspect herein, concerns the photo-reactive precursor solution of any of the 1^{st} to 12^{th} aspects, further comprising an additional agent.

A 14^{th} aspect of the present disclosure, either alone or in combination with any other aspect herein, concerns the photo-reactive precursor solution of the 13^{th} aspect, wherein the additional agent is chosen from a therapeutic agent, a microparticle comprised of a therapeutic agent and a biopolymer, a vitamin, an anti-coagulant, an anti-biotic, an anti-fungal, an anti-microbial, an anti-inflammatory, an anti-oxidant, a chemotherapeutic, an amino acid, a growth factor, fibrin, collagen, a stem cell, or any combination thereof.

A 15^{th} aspect of the present disclosure, either alone or in combination with any other aspect herein, concerns the photo-reactive precursor solution of the 14^{th} aspect, wherein the anti-biotic is chosen from phenoxymethylpenicillin, penicillin, dicloxacillin, amoicillin, ampicillin, nafcillin, oxacillin, penicillin V, penicillin G, cephalosporin, cefaclor, cefazolin, cefadroxil, cephalexin, cefoxime, cefixime, cefoxitin, ceftriaxone, tetracycline, doxycycline, minocycline, sarecycline, erythromycin, clarithromycin, azithromycin, fidaxomicin, roxithromycin, ciprofloxacin, ofloxacin, levofloxacin, moxifloxacin, sulfamethoxazole, sulfasalazine, sulfacetamide, sulfadiazine, vancomycin, dalbavancin, oritavancin, telavancin, or combinations thereof.

A 16^{th} aspect of the present disclosure, either alone or in combination with any other aspect herein, concerns the photo-reactive precursor solution of the 14^{th} aspect, wherein the concentration density of the therapeutic agent in the precursor solution or within the polymer microparticle is of from 0.1 μg/mm² to 10 μg/mm²

A 17^{th} aspect of the present disclosure, either alone or in combination with any other aspect herein, concerns the photo-reactive precursor solution of any of the 1^{st} to 16^{th} aspects, wherein the precursor solution further comprises one or more excipients.

An 18^{th} aspect of the present disclosure, either alone or in combination with any other aspect herein, concerns the photo-reactive precursor solution of any of the 1^{st} to 17^{th} aspects, wherein the monomer comprises HA and photo-initiator comprises riboflavin.

A 19^{th} aspect of the present disclosure, either alone or in combination with any other aspect herein, concerns the photo-reactive precursor solution of the 18^{th} aspect, further comprising DMAEMA.

A 20^{th} aspect of the present disclosure, either alone or in combination with any other aspect herein, concerns a method for filling a cavity within a subject comprising administering the precursor solution of any of aspects 1-19 to the cavity and applying a light source thereto.

A 21^{st} aspect of the present disclosure, either alone or in combination with any other aspect herein, concerns a method to fill a cavity in a subject comprising preparing a precursor solution as set forth in any of aspects 1-19; providing the precursor solution to a barrel of a syringe, wherein the barrel is operably connected to a needle and a plunger; depressing the plunger to extrude the precursor solution from the needle, wherein the needle is aimed into the cavity; and, applying a light source to the extruded precursor solution in the cavity.

A 22^{nd} aspect of the present disclosure, either alone or in combination with any other aspect herein, concerns the method of the 21^{st} aspect, wherein the light source is affixed to the syringe.

A 23^{rd} aspect of the present disclosure, either alone or in combination with any other aspect herein, concerns the method of the 22^{nd} aspect, wherein the light source is on a distal end of the needle.

A 24^{th} aspect of the present disclosure, either alone or in combination with any other aspect herein, concerns a syringe device for photocuring a gel, comprising a barrel operably connected to a needle and a plunger, and a light source at a distal end of the needle.

A 25^{th} aspect of the present disclosure, either alone or in combination with any other aspect herein, concerns the syringe device of the 24^{th} aspect, wherein the light source comprises light emitting diodes (LEDs) and fiber optic cables, and wherein the fiber optic cables run the length of the inner cannula of the needle.

A 26^{th} aspect of the present disclosure, either alone or in combination with any other aspect herein, concerns the syringe device of the 25^{th} aspect, wherein the LEDs are operably connected to a power source.

These and additional features provided by the embodiments described herein will be more fully understood in view of the following detailed description, in conjunction with the drawings.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings, where like structure is indicated with like reference numerals and in which:

FIG. 1A shows an embodiment of the syringe delivery device for application of the bioabsorbable gels described herein.

FIG. 1B shows an aerial cross-section of along line A-B of FIG. 1A.

FIG. 1C shows an aerial cross-section of along line C-D of FIG. 1A.

### DETAILED DESCRIPTION

The present disclosure concerns bioabsorbable gels with markers suspended therein that fill a surgical void in a patient. It is a facet of surgical procedures, particularly when tissue is resected, that a space or void is created or introduced prior to the closing of any incisions. For example, some cancer treatments include resection of malignant tissue to prevent metastasis or spreading to healthy tissue. These procedures leave a void or space within the subject in the area in and around where the resected tissue resided. In some aspects, the volume and/or location of the created space or void can cause a collapse of surrounding tissue due to lack of structural support. While the subject's body can regenerate some tissue to appease the space created, these processes are slow and often result in permanent deformation or dimpling at the surface. It is an aspect of the present disclosure, therefore, to provide a bioabsorbable gel to the space or void created within a subject, such as through surgical procedures, and provide support to the surrounding tissue and/or organs.

In some aspects of the present disclosure, the bioabsorbale gels are formed in situ in the void or space needed. In some aspects, the bioabsorbable gels are a polymer or combination of polymers that cross-link in situ. In some aspects, the bioabsorbable gels include a photo-reactive polymer or polymer pre-cursor solution that cross-links when exposed to light. In aspects, the precursor solution includes a monomer and a photo-initiator. In some aspects, the precursor solution includes a monomer, a photo-initiator, and an oligomer or macromolecule.

In some aspects, the precursor solution is prepared and provided to a syringe body, optimally without prior light exposure. In some aspects, the syringe body is of an opaque material to prevent light penetration. The precursor solution is then deployed out of the syringe body through the cannula of an operably attached needle and into the space or void to be filled in the patient. Once the precursor solution is in place, or as it is applied to the space or void, light is provided to initiate cross-linking and the gel forms in situ.

In some aspects, light may be provided to cross-link the gel from a natural source. In some aspects, a user applies or shines a light source directly on to the dispensed precursor solution. In certain aspects, the light source may be part of or connected to the dispensing syringe. In some aspects, surrounding the cannula with one or more fiber-optic cables that are operably connected to a light source allows for light to be provided to the precursor solution.

In some aspects, the present disclosure concerns the precursor solution and/or the resulting bioabsorbable gel produced by photo-initiated cross-linking thereof. In some aspects, the present disclosure concerns a solution of at least two polymer precursor units such as a monomer and an oligomer or macromolecule with a photo-initiator, wherein light energy is absorbed to the photo-initiator to catalyze the cross-linking of bonding and/or entanglement between reactive groups present in each precursor unit. In some aspects, the bioabsorable gel may include a homopolymer or a polymer having the same repeating monomeric unit throughout its structure. In some aspects, the bioabsorbable gel may include a copolymer or a polymer having at least two dissimilar repeating monomeric units throughout its structure which are distributed randomly or in a block fashion.

In some aspects, the bioabsorbable get or the precursor solution thereof includes one or more photocurable polymer chains or units of monomers. In some aspects, the biobaborbable polymer may include a precursor solution of monomers such as methacrylated gelatin, polyethylene glycol diacrylate (PEGDA), or a combination thereof. In some aspects, the bioabsorbable gel or precursor solution may include monomers of polyethylene glycol (PEG), polylactic acid (PLA), polyglycolic acid (PGA), polylactic-co-glycolic acid (PLGA), polycaprolactone (PCL), aloe vera gel, hyaluronic acid (HA), sodium hyaluronate, or combinations thereof. In some aspects, the bioabsorbable gel or precursor solution thereof may include methacrylated gelatin, polyethylene glycol diacrylate (PEGDA), or a combination thereof and at least one of PEG, PLA, PLGA, PGA, PCL, aloe vera gel, and sodium hyaluronate. In some aspects, the bioabsorbable gel or precursor solution thereof may include a-hydroxy carboxylic acids, dioxepanones, dioxanones, lactides, lactones and carbonates, lactide, glycolide, p-dioxanone, y-caprolactone, or combinations thereof.

In some aspects, the bioabsorbable gel or precursor solution thereof may include oligomers or macromolecules. Such may be added to become entangled in the cross-linking and further affect the rate of formation and/or elastic modulus and/or rheology and/or viscoelasticity. Examples of such include polymers or peptides or proteins, such as polyethyleneimine (PEI), poly-lysine, or albumin, including recombinant human serum albumin. (rHA).

In some aspects, the bioabsorbable gel or precursor solution thereof may include a tracker material, such that a user can locate the region or area of the formed bioabsorbable gel by identifying the presence of the tracker material. In some aspects, the tracker material is provided to the precursor solution prior to cross-linking initiation, such as at least before the precursor solution is deployed from the syringe. In some aspects, the tracker material may be added to the precursor solution in the cannula of the syringe. In some aspects, the tracker material may be added prior to application of light to the precursor solution. In some aspects, the tracker material may be added to the precursor solution during the polymerization. It will be apparent that the tracker material works best when incorporated within the formed bioabsorbable gel, thereby providing an accurate location of the gel itself rather than just the tracker material.

In some aspects, the tracker material may include titanium, nitinol, polyvinyl acetate (PVA), PEG, stainless steel (e.g. BioDur® 108) or combinations thereof. In some aspects, the tracker material may be an inert material. In some aspects, the tracker material may be of a slower bioabsorbable material than the gel. It will be appreciated that the tracker material may include materials that can be located through analytical techniques such as ultrasound, computed-tomography, magnetic resonance, x-ray, positron-emission tomography, sonography, and/or radiography.

In some aspects, the bioabsorbable gel or precursor solution thereof may include other materials suspended therein. It will be appreciated that including other materials in the precursor solution prior to cross-linking thereof allows for further materials to be suspended therein. Such other materials may include those that may assist in the healing or health of the subject, such as pharmaceutical or therapeutic agents, vitamins, anti-coagulants, anti-biotics, anti-fungals, anti-microbials, anti-inflammatoireanti-oxidants, chemotherapeutics, amino acids, growth factors, fibrin, collagen, stem cells, and combination thereof.

In some aspects, the texture of the bioabsorbable gel can be controlled by the concentration of polymer units or chains included in the precursor solution. In aspects, the bioabsorbable gels of the present disclosure can be set to achieve a desired rate of formation and/or elastic modulus and/or rheology and/or viscoelasticity. Factors such as the monomeric unit(s) selected, the concentration of the selected monomeric unit(s), the length of each monomer in the gel, additional macromolecule(s) selected, the concentration of the monomer molecule(s) and/or optional oligomer or macromolecule(s) selected, the degree of crosslinking, the available side chains for cross-linking, the length of time of light exposure, and so forth. For example, varying the concentrations and the MW of the monomer can affect the gelling time and/or the degradation time. Further, the addition of an oligomer or macromolecule can even further allow for the tenability of the former bioabsorbable gels. In some aspects, the texture of the bioabsorbable gel can be further controlled by control of the curing time or the amount of polymerization allowed to occur. In some aspects, the desired physical properties of the bioabsorbable gel may depend on the area within the subject it is being applied within. For example, if the bioabsorbable gel is to support an organ or to prevent two organs from contact, a more rigid gel may be appropriate. In some aspects, the bioabsorbable gel may be provided to more emulate softer tissue. In some aspects, the bioabsorbable gel may be placed near the skin, and as such may be emulate the surrounding tissue's own texture. It will further be appreciated that the gel provide sufficient support and structure, as well as volume, such that the skin does not dimple or sink into the space occupied by the gel as the underlying tissue heals and/or regenerates.

In some aspects, the bioabsorbable gel or precursor solution thereof includes a photo-initiator to cause the cross-linking to initiate. The concentration of the photo-initiator may also cause the rate of polymerization to vary. Examples of photo-initiators include riboflavin, dimethylaminoethyl methacrylate (DMAEMA), lithium acylphosphinate (LAP), 2,2-dimethoxy-1,2-diphenylethan-1-one, 2-hydroxy-2-methyl-1-phenylpropanone, 1-hydroxy-cyclohexylphenylketone, benzophenone, isopropyl thioxanthone, 2-ethylhexyl-(4-N,N-dimethyl amino) benzoate, ethyl-4-(dimethylamino) benzoate, azobisisobutyronitrile, benzoyl peroxide, camphorquinone, 2-hydroxy-2-methyl propiophenone, 2-hydroxy-4′-(2-hydroxyethoxy)-2-methyl propiophenone, methobenzoyl formate, diphenyl (2,4,6-trimethylbenzoyl)-phosphine oxide, phosphine oxide phenyl bis (2,4,69 trimethyl benzoyl), 2-methyl-1-[4-(methylthio)phenyl]-2-(4-morpholinyl)-1-propanone, 2-Benzyl-2-(dimethylamino)-1-[4-(4-morpholinyl) phenyl]-1-butanone, 2-Dimethylamino-2-(4-methyl-benzyl)-1-(4-morpholin-4-yl-phenyl)-butan-1-one, Bis(.eta.5-2,4-cylcopentadien-1-y1)-bis(2,6-difluoro-3-(1H-pyrrol-1-yl)-Phenyl)titanium, 2-Ethyl Anthraquinone, 2,4-diethyl thioxanthone, Benzil Dimethyl Ketal, 4-chloro Benzophenone, methyl-2-benzoylbenzoate, 4-phenyl Benzophenone, 2,2′-Bis(2-Chlorophenyl)-4,4′, 5,5′-Tetraphenyl-1,2′-Bi-imidazole, 2,2′,4-tris(2-chlorophenyl)-5-(3,4-dimethoxyp-enly)-4′5′-diphenyl-1,1′-biimidazole, 4-phenoxy-2.′2′-Dichloro Acetophenone, Ethyl-4-(dimethylamino)Benzoate, isoamyl 4-(Dimethylamino)benzoate, 2-Ethyl Hexyl-4-(dimethylamino) Benzoate, 4,4′Bis(diethylamino) Benzophenone, 4-(4′-methylphenylthio)-benzophenone, 1,7-Bis(9-acridinyl)heptane, N-phenyl glycine, a ruthenium metal complex and sodium persulfate, Irgacure 2959, Irgacure 184, Irgacure 651, Irgacure 369, Irgacure 907, monoacylphosphine (TPO), TPO-sodium or lithium, bisacylphosphine oxide or slats thereof, VA-086, eosin-Y, riboflavin, WSPI, BDEA, P2CK, rose Bengal, erythrosine, diphenyliodonium hexafluorophosphate, bis-(4-t-butylphenyl) iodonium hexafluorophosphate, N-methyldiethanolamine, or similar or combinations thereof.

In some aspects, the photo-initiator is present in the precursor solution at a concentration of about 0.01 to about 0.4% w/v, including about 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, and 0.39 % w/v.

In some aspects, an accelerator co-initiator may be utilized to quicken the rate of gel formation. In some aspects, an initiator may act as an accelerator or coinitiaiator with another photoinitiator compound. In some aspects, such may include diphenyliodonium, iodonium salt(s), thioxantone, iodine salt, dialkyl aminobenzoic acid, benzyl alcohol(s), tetramethylethylenediamine (TEMED), or similar.

In some aspects, the bioabsorbable gel or precursor solution thereof is of from about 0.1 to about 70 percent by weight of the monomer units, including about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69 percent by weight of the bioabsorbable gel. In some aspects, the bioabsorbable gel is of from about 0.1 to 70% by weight of methacrylated gelatin, polyethylene glycol diacrylate (PEGDA), or a combination thereof. In some aspects, the bioabsorbable gel is of from about 0.1 to 70% by weight of PEG, PLA, PGA, PLGA, PCL, aloe vera gel, sodium hyaluronate, or combinations thereof. In some aspects, the bioabsorbable gel is of from about 0.1 to 70% by weight of methacrylated gelatin, polyethylene glycol diacrylate (PEGDA), PEG, PLA, PLGA, PGA, PCL, aloe vera gel, sodium hyaluronate, or any combination thereof. In some aspects, In some aspects, the oligomer or macromolecule has a molecular weight of from about 1,000 Da to about 100,000 Da. For example, rHSA has a MW of about 66 kDa. PEI has a variable MW depending on the length of the monomer. Similarly, a poly-lysine polypeptide MW can be constructed based on a desired size. In some aspects, the oligomer or macromolecule is provided within the gel at about 2 to about 60% weight/volume (w/v), including about 3, 4, 5, 6, 7, 8, 9, 10, 15, 20, 25, 30, 35, 40, 45, 50, and 55% w/v. In some aspects, the oligomer or macromolecule is of from about 5 to about 50% w/v, from about 5 to about 40% w/v, from about 5 to about 30% w/v, from about 5 to about 20% w/v, from about 5 to about 10% w/v, from about 10 to about 50% w/v, from about 10 to about 40% w/v, from about 10 to about 30 % w/v, from about 10 to about 20% w/v, from about 20 to about 50% w/v, from about 20 to about 40% w/v, from about 20 to about 30% w/v, from about 30 to about 50% w/v, from about 30 to about 40% w/v, or from about 40 to about 50% w/v. In some aspects, the oligomer or macromolecule is albumin (including human albumin) and/or PEI and/or poly-lysine.

In certain aspects, the precursor solution includes HA and a photoinitiator, such as riboflavin and/or DMAEMA. In some aspects, LAP is used as a phottoinitiator.

In some aspects, the precursor solution is cured or allowed to cross-link in situ through the application of light thereto. In some aspects, the light applied covers the visible spectrum. In some aspects the light applied covers a certain wavelength or range of wavelength within the visible spectrum that spans over about 5, 10, 15, 20, 25, 30, 35, 40, 50, 60, 70, 80, 90, 100, 150, 200, 250, 300, 350, 400, 450, 500, 550, 600, or 650 nm. In some aspects, the time of light application will allow the user to control or partially control the formation of the gel. In some aspects, the light is applied as the precursor solution is applied, allow for cross-linking to initiate as the solution settles in the desired space. In some aspects, the light is applied after the precursor solution is in place. In some aspects, the bioabsorbable gel is formed through multiple steps, such as by allowing a first volume of precursor into the space, followed by application of light, followed by a second volume and a second application of light, repeating as needed. It will further be appreciated that the steps for curing the precursor solution can be varied and/or adapted due to the volume of the space and/or the desired texted of the final bioabsorbable gel. In some aspects, the light is applied for a period of time from about 2 to about 60 seconds, including about 5, 10, 15, 20, 25, 30, 35, 40, 45, 50, and 55 seconds. In some aspects, light may need to be applied for a longer period. In some aspects, light may be applied in several applications.

In aspects, the precursor solution includes one or more therapeutics therein, such that the therapeutics are embedded within the polymer as it cures during exposure to light. layer includes one or more therapeutic agents. In some aspects, the therapeutic agent is chosen from paclitaxel, rapamycin, daunorubicin, 5-fluorouracil, doxorubicin, sunitinib, sorafenib, irinotecan, bevasizumab, cetuxamab, biolimus (biolimus A9), everolimus, sirolimus, zotarolimus, tacrolimus, dexamethasone, prednisolone, corticosterone, cisplatin, vinblastine, lidocaine, bupivacaine, or a combination thereof.

In aspects, the therapeutic may include an anti-coagulant therapeutic. Such may include warfarin, apixaban, rivaroxaban, edoxaban, dabigatran, or combinations thereof.

In aspects, the therapeutic agent may include an antiobiotic, such as a pencillin, a glycopeptide, a sulfonamide, a fluoroquinolone, a macrolide, a tetracycline, a cephalosporin, or a combination thereof. Examples include phenoxymethylpenicillin, penicillin, dicloxacillin, amoicillin, ampicillin, nafcillin, oxacillin, penicillin V, penicillin G, cephalosporin, cefaclor, cefazolin, cefadroxil, cephalexin, cefoxime, cefixime, cefoxitin, ceftriaxone, tetracycline, doxycycline, minocycline, sarecycline, erythromycin, clarithromycin, azithromycin, fidaxomicin, roxithromycin, ciprofloxacin, ofloxacin, levofloxacin, moxifloxacin, sulfamethoxazole, sulfasalazine, sulfacetamide, sulfadiazine, vancomycin, dalbavancin, oritavancin, telavancin, or combinations thereof.

In some aspects, the gel is applied to resected tumorous tissue, such as aa cancerous or pre-cancerous tissue. In such instances, it may be advantageous to include a chemotherapeutic to treat the surround tissue to ensure efficacy in targeting cancerous cells. In some aspects, the bioabsorbable gel or precursor solution thereof may therefore include a therapeutic for cancers such as breast or prostate cancer. In some aspects, the therapeutic may include one or more of docetaxel, degarelix, abiraterone, apalutamide, bicalutamide, cabazitaxel, darolutamide, leuprolide, enzalutamide, flutamide, goserelin, lutetium Lu¹⁷⁷ vipivotide, tetraxetan, olaparib, mitoxantrone, nilutamide, relugolix, sipuleucel-T, radium 223, rucaparib camsylate, raloxifene, tamoxifene, abemaciclib, paclitaxel, trastuzumab, everolimus, alpelisib, anastrozole, pamidronate, exemestane, capecitabine, cyclophosphamide, docetaxel, doxorubicin, elacestrant, epirubicin, eribulin mesylate, fluorouracil, toremifene, fulvestrant, letrozole, gemcitabine, goserelin, palbociclib, ixabepilone, pembrolizumab, ribociclib, lapatinib, olaparib, margetuximab, megestrol, methotrexate, neratinib, pertuzumab, sacituzumab, talazoparib, thiotepa, tucatinib, vinblastine, paclitaxel, rapamycin, daunorubicin, 5-fluorouracil, doxorubicin, sunitinib, sorafenib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, triamciclone, tranilast, halofuginone, montelukast, zafirlukast, pirfenidone, nintedanib, avapritinib, abemaciclib, erdafitinib, fedratinib, nilotinib, nintendanib, palbociclib, pemigatinib, xanthines, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, theophylline, resveratrol, quercetin, curcumin, chrysin, myricetin, luteolin, apigenin, anthrocyanin, genistein, epigallocatechin gallate, fisetin, astxanthin, tetrahydrocurcumin, imatinib, nintedanib, sorafenib, sunitinib, pazopanib, ROCK inhibitor (Y27632), YAP/TAZ inhibitor (CA3 and verteporfin), YAP/TAZ-TEAD interaction inhibitor (verteporfin, VGLL4 peptide), and SRC inhibitor (dasatinib).

The therapeutic may be dissolved in the precursor solution or suspended therein in an amorphous or crystalline form. In some aspects, the crystalline and/or amorphous therapeutic agent size can vary from about 0.1 μm to about 100 μm, including about 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 99 μm and any size or number therein. In some aspects, the particle size is of from about 1 μm to about 20 μm. In other aspects, the particle size of from about 10 μm to about 100 μm. Size selection can be achieved through methods understood in the art, such as by passing through mesh of a pre-determined pore or hole size. The desired particle size can be achieved by dry grind or wet grinding. The grinding method may include techniques such as use of a jaw crusher, ultra-centrifugal mill, cyclone mill, cross beater mill, rotor beater mill, cutting mill, knife mill, mortar grinder, disc mill, mixer mill, cryomill, planetary ball mill, drum mill, and/or fine grinding rod mill.

In some aspects, the precursor solution may include microparticles of therapeutic agent(s) and a bioabsorbable polymer. As with the therapeutics, the microparticles become embedded within the cured polymer following light exposure. Microparticles may be prepared the evaporation of a solvent with a bioabsorbable/biodegradable polymer and at least one therapeutic therein. In some aspects, the solvent is of dichloromethane (DCM) or ethyl acetate (EtOAc). Polymers may include a network of a poly-glycolic acid (PGA) and a poly-L-lactic acid (PLLA). Other bioabsorbable polymers that can be utilized in combination or alone for the microparticles include polycaprolactone (PCL), poly-DL-lactic acid (PDLLA), poly(trimethylene carbonate) (PTMC), poly (ester amine)s (PEA), poly(para-dioxanone) (PPDO), poly-2-hydroxy butyrate (PHB), and co-polymers with various ratios thereof. In some aspects, the bioabsorbable polymer may include, either alone or in combination with other bioabsorbable polymers, a polymer combination of lactic acid and glycolic acid, poly-lactic-co-glycolic acid (PLGA). Those skilled in the art will appreciate that PLGA can be of varying percentages of lactic acid and glycolic acid, wherein the higher the amount of lactide units, the longer the polymer can last in situ before degrading. Additional tunable properties with PLGA concern the molecular weight, with higher weights showing increased mechanical strength. In some aspects, the polymer microparticle is also loaded or embedded with an antioxidant, such as BHT.

In some aspects, the concentration density of the therapeutic agent in the precursor solution or within the polymer microparticle may be from 0.1 μg/mm² to 10 μg/mm², from 0.1 μg/mm² to 8 μg/mm² , from 0.1 μg/mm² to 6 μg/mm², from 0.1 g/mm² to 4 μg/mm², from 0.1 μg/mm² to 2 μg/mm², from 0.1 μg/mm² to 1 μg/mm², from 1 μg/mm² to 10 μg/mm², from 1 μg/mm² to 8 μg/mm², from 1 μg/mm² to 6 μg/mm², from 1 μg/mm² to 4 μg/mm², from 1 μg/mm² to 2 μg/mm², from 2 μg/mm² to 10 μg/mm², from 2 μg/mm² to 8 μg/mm², from 2 μg/mm² to 6 μg/mm², from 2 μg/mm² to 4 μg/mm², from 4 μg/mm² to 10 μg/mm², from 4 μg/mm² to 8 μg/mm², from 4 μg/mm² to 6 μg/mm², from 6 μg/mm² to 10 μg/mm², from 6 μg/mm² to 8 μg/mm², or from 8 μg/mm² to 10 μg/mm². In some aspects the concentration density of therapeutic agent in the precursor solution or polymer microparticle may be from 0.5 μg/mm² to 5 μg/mm².

In some aspects, the precursor solution includes one or more excipients. Suitable excipients that can be used in some aspects of the present disclosure include, without limitation, organic and inorganic pharmaceutical excipients, natural products and derivatives thereof (such as sugars, vitamins, amino acids, peptides, proteins, and fatty acids), surfactants (anionic, cationic, non-ionic, and ionic), and mixtures thereof. The following list of excipients useful in the present disclosure is provided for exemplary purposes only and is not intended to be comprehensive. Many other excipients may be useful for purposes of the present disclosure, such as polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, polysorbates, polyethylene glycol, polyvinylpyrrolidone (PVP) and aliphatic polyesters.

In some aspects, the one or more additional excipients may include an antioxidant. An antioxidant is a molecule capable of slowing or preventing the oxidation of other molecules. Oxidation reactions can produce free radicals and/or peroxides, which start chain reactions and may cause degradation of therapeutic agents. Antioxidants terminate these chain reactions by removing free radicals and inhibiting oxidation of the active agent by being oxidized themselves. Antioxidants are used as the one or more additional excipients in certain aspects to prevent or slow the oxidation of the therapeutic agents in the coatings for medical devices. Antioxidants are a type of free radical scavengers. The antioxidant may be used alone or in combination with other additional excipients in certain aspects and may prevent degradation of the active therapeutic agent during sterilization or storage prior to use. Some representative examples of antioxidants that may be used in the drug coatings of the present disclosure include, without limitation, oligomeric or polymeric proanthocyanidins, polyphenols, polyphosphates, polyazomethine, high sulfate agar oligomers, chitooligosaccharides obtained by partial chitosan hydrolysis, polyfunctional oligomeric thioethers with sterically hindered phenols, hindered amines such as, without limitation, p-phenylene diamine, trimethyl dihydroquinolones, and alkylated diphenyl amines, substituted phenolic compounds with one or more bulky functional groups (hindered phenols) such as tertiary butyl, arylamines, phosphites, hydroxylamines, and benzofuranones. Also, aromatic amines such as p-phenylenediamine, diphenylamine, and N, N′ disubstituted p-phenylene diamines may be utilized as free radical scavengers. Other examples include, without limitation, butylated hydroxytoluene (“BHT”), butylated hydroxyanisole (“BHA”), L-ascorbate (Vitamin C), Vitamin E, herbal rosemary, sage extracts, glutathione, resveratrol, ethoxyquin, rosmanol, isorosmanol, rosmaridiphenol, propyl gallate, gallic acid, caffeic acid, p-coumeric acid, p-hydroxy benzoic acid, astaxanthin, ferulic acid, dehydrozingerone, chlorogenic acid, ellagic acid, propyl paraben, sinapic acid, daidzin, glycitin, genistin, daidzein, glycitein, genistein, isoflavones, and tertbutylhydroquinone. Examples of some phosphites include di(stearyl)pentaerythritol diphosphite, tris(2,4-di-tert. butyl phenyl)phosphite, dilauryl thiodipropionate and bis(2,4-di-tert. butyl phenyl)pentaerythritol diphosphite. Some examples, without limitation, of hindered phenols include octadecyl-3,5,di-tert.butyl-4-hydroxy cinnamate, tetrakis-methylene-3-(3′,5′-di-tert.butyl-4-hydroxyphenyl)propionate methane 2,5-di-tert-butylhydroquinone, ionol, pyrogallol, retinol, and octadecyl-3-(3,5-di-tert.butyl-4-hydroxyphenyl)propionate. An antioxidant may include glutathione, lipoic acid, melatonin, tocopherols, tocotrienols, thiols, Beta-carotene, retinoic acid, cryptoxanthin, 2,6-di-tert-butylphenol, propyl gallate, catechin, catechin gallate, and quercetin. Preferable antioxidants are butylated hydroxytoluene (BHT) and butylated hydroxyanisole (BHA).

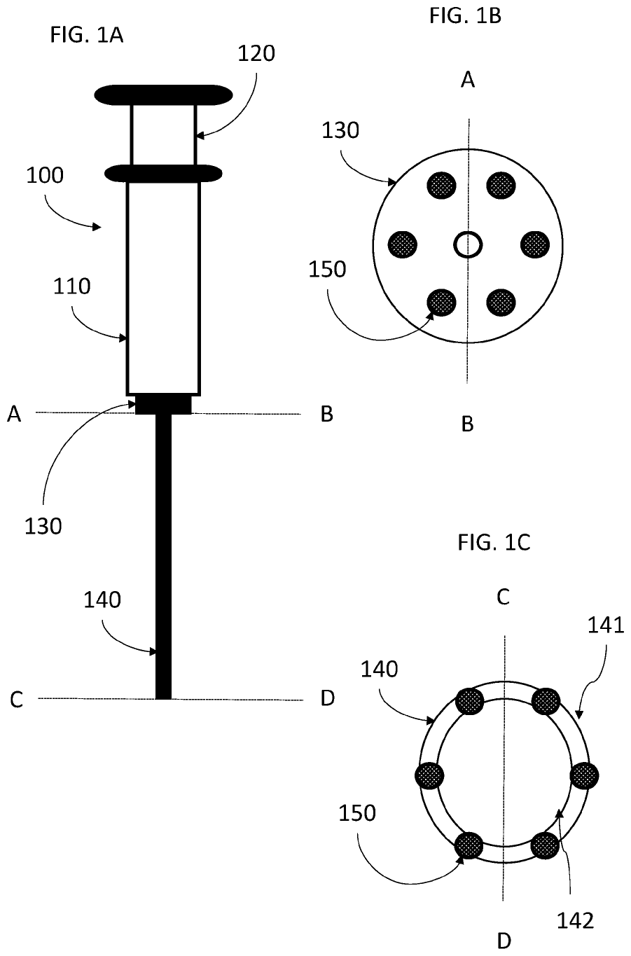

In some aspects, the present disclosure provides for a device to cure the precursor solution in situ within the subject. Turning to FIG. 1A, depicted is a side-view embodiment of such a syringe device 100 that includes a barrel 110, a plunger 120, a hub 130, and a needle 140. The precursor solution is stored within the barrel 110 and when used the plunger 120 is depressed into the cavity of the barrel 110 and the precursor solution flows into a desired space from the end of the needle 140. As depicted in FIGS. 1B and 1C, lights 150 can be present on the hub 130 or the end of the needle 140. FIG. 1B shows an aerial view along line A-B of FIG. 1A, where lights 150 are dispersed around the outer surface of the hub 130. The lights 150 can be of a fiber optic LED or an LED bulb, either of any spectrum or desired wavelength of light. FIG. 1C shows an aerial view along line C-D of the end of the needle 140 in FIG. 1A. Between the outer cannula 141 and the inner cannula, lights 150 surround the opening of the needle 140 to allow for light to contact the precursor solution as it leaves the end of the needle 140 of the syringe device 100. Fiber optic lines or cables may run along the length of the inner cannula 142 or outer cannula 141 or therebetween to operably connect the lights 150 to a light source.

In aspects, the present disclosure concerns methods of forming the bioabsorbable gel in situ in a desired space or cavity of a subject. In some aspects, the methods concerns applying a precursor solution or portion thereof to a cavity in a subject and applying light energy therein to initiate cross-linking and the formation of the bioabsorable gel. In some aspects, light may be applied as the solution pours or reaches the cavity. In some aspects, light may be intermittently applied after a portion of the precursor solution has filled the cavity. In some aspects, light may be applied after the full volume of the precursor solution is in the cavity.

In some aspects, as the gel begins to cure, the cross-linking therein produces or releases heat. Such exothermic properties can provide additional benefits to the subject such as sterilization of the cavity and/or cauterization.

In some aspects, the methods further include closing external access to the cavity or space, such as through suturing the subject's skin. In aspects, the method may include monitoring the formed bioabsorbable gel over time. As described herein, the gel will eventually degrade within the subject. In some aspects, the gel is tuned to degrade at or near the rate of tissue formation, such that the subject's own tissue fills the space or cavity at or near the same rate as the bioabsorbale gel erodes.

In some aspects, the methods include the controlled release of materials from within the biobabsorable gel as it erodes. As described herein, the precursor solution may include additional materials, such as therapeutics or microparticles. Such materials can be suspended within the solution as the gel is introduced and cured in the space or cavity. Such allows for the additional materials to be suspended within the formed gel. As the gel then erodes or is absorbed by the subject over time, the materials therein are released and can interact or absorb with the subject's tissue.

In some aspects, the methods include leaving a permanent or semi-permanent marker within the subject. As described herein, the precursor solution may include a tracker material. Such tracker material will not erode or be bioabsorbed or will erode or be bioabsorbed at a slower rate than the gel. Accordingly, the tracker material will reside in the (former) space or cavity after the gel has been absorbed, thereby allowing a user to identify the presence of the tracker material and hence monitor the former cavity or space, such as evaluating the health of the tissue, both surrounding the former space or cavity, as well as the new tissue that filled therein.

### EXAMPLES

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

1. A photo-reactive precursor solution of a bioabsorbable gel comprising a monomer and a photo-initiator.

2. The photo-reactive precursor solution of claim 1, further comprising an oligomer selected from polyethyleneimine (PEI), poly-lysine, and serum albumin.

3. The photo-reactive precursor solution of claim 1, wherein the monomer is selected from methacrylated gelatin, polyethylene glycol diacrylate (PEGDA), or a combination thereof.

4. The photo-reactive precursor solution of claim 3, further comprising polyethylene glycol (PEG), polylactic acid (PLA), polyglycolic acid (PGA), polylactic-co-glycolic acid (PLGA), polycaprolactone (PCL), aloe vera gel, hyaluronic acid (HA), sodium hyaluronate, or a combinations of two or more thereof.

5. The photo-reactive precursor solution of claim 1, further comprising an a-hydroxy carboxylic acid, a dioxepanone, a dioxanones, a lactide, a lactone, a carbonate, a glycolide, p-dioxanone, y-caprolactone, or a combination of two or more thereof.

6. The photo-reactive precursor solution of claim 1, further comprising a tracker material.

7. The photo-reactive precursor solution of claim 6, wherein the tracker material is selected from titanium, nitinol, polyvinyl acetate (PVA), PEG, stainless steel, or combinations thereof.

8. The photo-reactive precursor solution of claim 1, wherein the monomer comprises 0.1 to 70 percent by weight of the precursor solution.

9. The photo-reactive precursor solution of claim 1, wherein the photo-initiator is chosen from riboflavin, dimethylaminoethyl methacrylate (DMAEMA), lithium acylphosphinate (LAP), 2,2-dimethoxy-1,2-diphenylethan-1-one, 2-hydroxy-2-methyl-1-phenylpropanone, 1-hydroxy-cyclohexylphenylketone, benzophenone, isopropyl thioxanthone, 2-ethylhexyl-(4-N,N-dimethyl amino) benzoate, ethyl-4-(dimethylamino) benzoate, azobisisobutyronitrile, benzoyl peroxide, camphorquinone, 2-hydroxy-2-methyl propiophenone, 2-hydroxy-4′-(2-hydroxyethoxy)-2-methyl propiophenone, methobenzoyl formate, diphenyl (2,4,6-trimethylbenzoyl)-phosphine oxide, phosphine oxide phenyl bis (2,4,69 trimethyl benzoyl), 2-methyl-1-[4-(methylthio)phenyl]-2-(4-morpholiny1)-1-propanone, 2-Benzyl-2-(dimethylamino)-1-[4-(4-morpholinyl) phenyl]-1-butanone, 2-Dimethylamino-2-(4-methyl-benzyl)-1-(4-morpholin-4-yl-phenyl)-butan-1-one, Bis(.eta.5-2,4-cylcopentadien-1-yl)-bis(2,6-difluoro-3-(1H-pyrrol-1-yl)-Phenyl)titanium, 2-Ethyl Anthraquinone, 2,4-diethyl thioxanthone, Benzil Dimethyl Ketal, 4-chloro Benzophenone, methyl-2-benzoylbenzoate, 4-phenyl Benzophenone, 2,2′-Bis(2-Chlorophenyl)-4,4′, 5,5′-Tetraphenyl-1,2′-Bi-imidazole, 2,2′,4-tris(2-chlorophenyl)-5-(3,4-dimethoxyp-enly)-4′5′-diphenyl-1,1′-biimidazole, 4-phenoxy-2.′2′-Dichloro Acetophenone, Ethyl-4-(dimethylamino)Benzoate, isoamyl 4-(Dimethylamino)benzoate, 2-Ethyl Hexyl-4-(dimethylamino) Benzoate, 4,4′Bis(diethylamino) Benzophenone, 4-(4′-methylphenylthio)-benzophenone, 1,7-Bis(9-acridinyl)heptane, N-phenyl glycine, a ruthenium metal complex and sodium persulfate, Irgacure 2959, Irgacure 184, Irgacure 651, Irgacure 369, Irgacure 907, monoacylphosphine (TPO), TPO-sodium or lithium, bisacylphosphine oxide or slats thereof, VA-086, eosin-Y, riboflavin, WSPI, BDEA, P2CK, rose Bengal, erythrosine, diphenyliodonium hexafluorophosphate, bis-(4-t-butylphenyl) iodonium hexafluorophosphate, N-methyldiethanolamine, or a combinations thereof.

10. The photo-reactive precursor solution of claim 1, wherein the photo-initiator comprises between 0.01 and 0.4% w/v of the precursor solution.

11. The photo-reactive precursor solution of claim 1, further comprising an accelerator.

12. The photo-reactive precursor solution of claim 11, wherein the accelerator is selected from diphenyliodonium, iodonium salt(s), thioxantone, iodine salt, dialkyl aminobenzoic acid, benzyl alcohol(s), tetramethylethylenediamine (TEMED), or a combination thereof.

13. The photo-reactive precursor solution of claim 1, further comprising an additional agent.

14. The photo-reactive precursor solution of claim 13, wherein the additional agent is chosen from a therapeutic agent, a microparticle comprised of a therapeutic agent and a biopolymer, a vitamin, an anti-coagulant, an anti-biotic, an anti-fungal, an anti-microbial, an anti-inflammatory, an anti-oxidant, a chemotherapeutic, an amino acid, a growth factor, fibrin, collagen, a stem cell, or any combination thereof.

15. The photo-reactive precursor solution of claim 14, wherein the anti-biotic is chosen from phenoxymethylpenicillin, penicillin, dicloxacillin, amoicillin, ampicillin, nafcillin, oxacillin, penicillin V, penicillin G, cephalosporin, cefaclor, cefazolin, cefadroxil, cephalexin, cefoxime, cefixime, cefoxitin, ceftriaxone, tetracycline, doxycycline, minocycline, sarecycline, erythromycin, clarithromycin, azithromycin, fidaxomicin, roxithromycin, ciprofloxacin, ofloxacin, levofloxacin, moxifloxacin, sulfamethoxazole, sulfasalazine, sulfacetamide, sulfadiazine, vancomycin, dalbavancin, oritavancin, telavancin, or combinations thereof.

16. The photo-reactive precursor solution of claim 14, wherein the concentration density of the therapeutic agent in the precursor solution or within the polymer microparticle is of from 0.1 μg/mm² to 10 μg/mm²

17. The photo-reactive precursor solution of claim 1, wherein the precursor solution further comprises one or more excipients.

18. The photo-reactive precursor solution of claim 1, wherein the monomer comprises HA and photo-initiator comprises riboflavin.

19. The photo-reactive precursor solution of claim 18, further comprising DMAEMA.

20. A method for filling a cavity within a subject comprising administering the precursor solution of claim 1 to the cavity and applying a light source thereto.

21-26. (canceled)
